(19) United States
(12) Patent Application Publication
(10) Pub. No.: US 2022/0375545 A1
(43) Pub. Date: Nov. 24, 2022

(54) SYSTEMS AND METHODS FOR GENERATING AND ANALYZING A CUSTOMIZED GENOMIC SEQUENCE INCORPORATING GENE FUSIONS FOR THERAPEUTIC APPLICATIONS

(71) Applicant: Arizona Board of Arizona, Tech Transfer Arizona, Tucson, AZ (US)

(72) Inventor: Adam David Grant, Tucson, AZ (US)

(21) Appl. No.: 17/747,817

(22) Filed: May 18, 2022

Related U.S. Application Data
(60) Provisional application No. 63/190,204, filed on May 18, 2021.

(51) Int. Cl. G16B 30/10; G16B 20/20

(52) U.S. Cl. CPC G16B 30/10; G16B 20/20

(57) ABSTRACT

Systems and methods are described for genetic analysis. In certain embodiments, the system reads a plurality of input parameters, where the input parameters comprise a path to a gene fusion input file and stores the gene fusion input file. The gene fusion input file is comprised of break points of genetic sequences for one or more gene fusion events. The computer then receives data identifying chromosome location, start position, end position, and strand for each gene in the gene fusion input file and loads a standardized reference genome. The computer then compares the gene fusion input file to the standardized reference genome and generates a gene fusion index file. The gene fusion index file identifies gene fusion events in the customized reference genome and can be used to quantify the number or next generation sequencing reads aligned to the wild type allele and fused allele. Allelic expression of tumor fusions can be used to diagnose a genetic condition and enhance therapeutic options for cancer patients.

### CROSS-REFERENCE TO RELATED APPLICATIONS

This application claims the benefit of U.S. Provisional Application No. 63/190,204, filed May 18, 2021, the contents of which are hereby incorporated by reference herein.

STATEMENT REGARDING FEDERALLY FUNDED RESEARCH

This invention was made with government support under Grant No. CA211878 awarded by the National Institutes of Health. The government has certain rights in the invention.

### FIELD OF THE INVENTION

The present invention relates to computer-implemented systems and methods for generating and analyzing customized genomic sequences that incorporate gene fusions, which are used for therapeutic applications.

### BACKGROUND OF THE INVENTION

Diseases like cancer can find their origins in mutations in the genetic sequences of cells. Sequencing is the process of determining the nucleic acid sequence—the order of nucleotides in the DNA or RNA of cells. It includes any method or technology that is used to determine the order of the four bases: adenine, guanine, cytosine, and thymine/uracil. As cancer is a genetic disease driven by heritable or somatic mutations, new DNA sequencing technologies will have a significant impact on the detection, management and treatment of disease.

DNA sequencing for use in cancer diagnostics may be computer-assisted. For example, next-generation sequencing may be used to catalogue gene fusions in multiple cancer types that can be translated to diagnostic, prognostic and therapeutic targets. However, as the technology is in its infancy, there is a need in the art for a system that has the capability to increase alignment sensitivity of predefined gene fusions, which can be used to improve detection of circulating cancer cells and enhance personalized therapeutic approaches for cancer patients.

### SUMMARY OF THE INVENTION

It is one object of the invention to disclose a system for genetic analysis that reads a plurality of input parameters, where the input parameters comprise a path to a gene fusion input file and stores the gene fusion input file. The gene fusion input file is comprised of breaks points of genetic sequences for one or more gene fusion events. The computer then receives data identifying chromosome location, start position, end position, and strand for each gene in the gene fusion input file and loads a standardized reference genome. The computer then compares the gene fusion input file to the standardized reference genome and generates a gene fusion index file. The gene fusion index file identifies gene fusion events in the customized reference genome and can be used to diagnose a genetic condition like cancer.

It is another object of the invention to remove duplicates if gene fusion events in the gene fusion input file are duplicated.

It is yet another object of the invention to check the user's fusion input file by matching non-altered nucleotides to the input reference genome.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the hardware of the genomic analysis system;

FIG. 2 is a flowchart detailing an exemplary process by which the software of the present invention operates; and

FIG. 3 is a flowchart detailing another exemplary process by which the software of the present invention operates.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

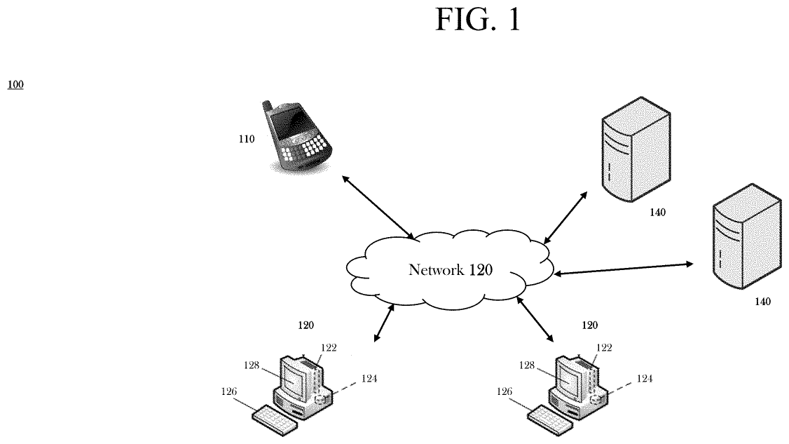

FIG. 1 is an exemplary embodiment of the genetic analysis system. In the exemplary system 100, one or more peripheral devices 110 are connected to one or more computers 120 through a network 130. Examples of peripheral devices 110 include smartphones, servers with databases that contain genetic/genomic data, and any other devices that can be used to collect genetic data that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 110 and each of the computers 120 of the system may have software related to the system installed on it. In such an embodiment, system data may be stored locally on the networked computers 120 or alternately, on one or more remote servers 140 that are accessible to any of the peripheral devices 110 or the networked computers 120 through a network 130. In alternate embodiments, the software runs as an application on the peripheral devices 110.

In certain embodiments, the software of the present invention is comprised of a script, referred to herein as “MAXX_Fusion.py” or “MAXX Fusion,” which generates a customized reference genome and an accompanying gene fusion index file based on a user's input of gene fusions, a reference genome, and GTF file. Customized reference genomes have been shown to improve alignment of next generation sequencing reads that contain a predefined gene fusion. This capability to increase alignment sensitivity of predefined gene fusions can potentially be used to improve detection of circulating cancer cells and enhance personalized therapeutic approaches for cancer patients.

To generate a gene fusion, the software combines the wildtype version of two genes. That is achieved by “cutting” each wild type gene where the breakpoint is identified by the user. Because a gene can be either on the plus strand or the minus strand of DNA, there are four ways that genes can be combined, as listed below:

1. 5′ plus+3′ plus=Gene1[start:break]+Gene2[break:end]

2. 5′ minus+3′ minus=Gene2[start:break]+Gene1 [break:end]

3. 5′ plus+3′ minus=Gene1[start:break]+Reverse(Gene2[start:break])

4. 5′ minus+3′ plus=Reverse(Gene2[break:end]) +Gene1 [break: end]

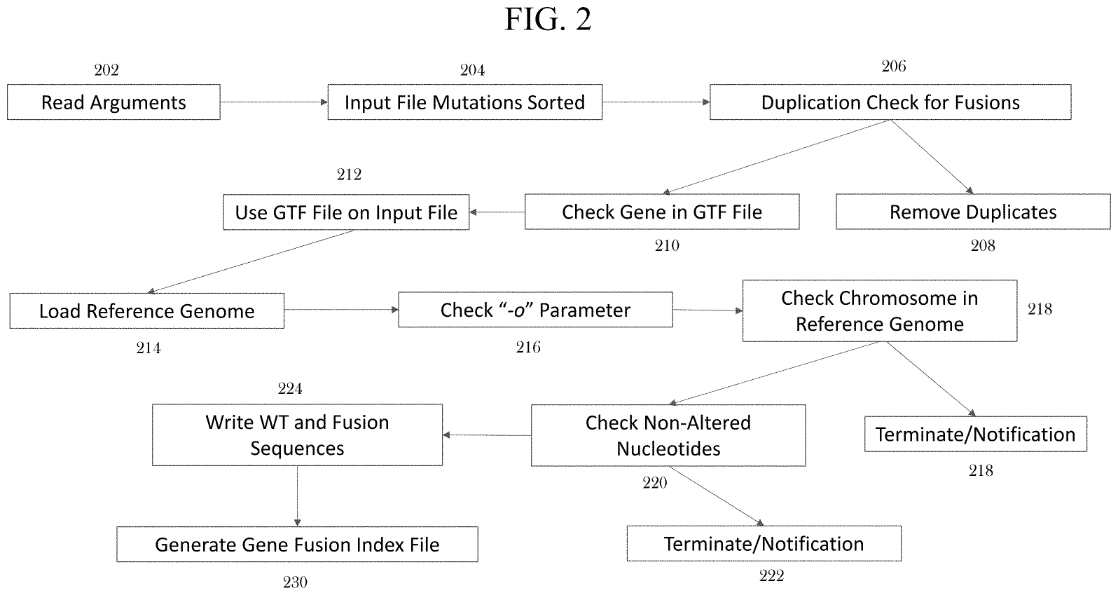

FIG. 2 is a flowchart detailing an exemplary process by which the software of the present invention operates. In general, the software, generates a customized reference genome based on a user's input of gene fusions.

At the step “Read Arguments,” 202, the MAXX Fusion script will read in arguments from the command line. In certain embodiments, MAXX Fusion has a total of 7 input parameters, but only 4 of them are required. Those exemplary required parameters are as follows: (1) -gf (required) is the path to the user's gene fusion input and is the list of gene fusions that the user wants the software to generate a customized reference genome for; (2) -f (required) is the path to the input reference genome; (3) -g (required) is the path to the input GTF file; (4) -s (required) is the name that will be associated with the output files; The optional parameters are: (5) -t DNA, -t gene, or -t transcript lets the user decide the nucleotide format for the fusion in the newly generated reference genome. -t DNA extends the fusion in the 3′and 5′direction based on the nucleotide padding parameter (-p), -t gene outputs the whole combined gene sequence of the fusion (default), and -t transcript outputs the whole combined transcript sequence of the fusion. (6) -o append or -o genes (optional) lets the user decided if the MAXX Fusion output reference genome should only contain the wild type and fused gene sequence of genes/transcripts from the gene/transcript fusion input file (default) or if MAXX Fusion should append the fused gene/transcript sequences to the input reference genome (-o append), or generate a reference genome that contains all gene/transcript sequences from the GTF file and the fused gene/transcript sequences (-o genes); and (7) -p number (optional) lets the users extend the length of the output gene fusion sequence and/or wild type sequence by adding corresponding nucleotides to the 5′and 3′ end of the genomic sequence.

At the step “Input File Stored,” 204, gene fusions from the gene fusion input file are put into a dictionary. At the step “Duplication Check for Fusions,” 206, the software checks if any gene fusions are duplicated in the gene fusion index file. If so, at step “Remove Duplicates” 208, the software removes duplicated gene fusions. At the step “Use GTF File on Input File,” 210, the software uses the GTF file to identify the chromosome location, start position, end position, and strand for each gene in the gene fusion input file. In certain embodiments, the gene fusion input file is comprised of break points of two genes mutated for genetic sequence comprised of one or more gene fusion events. An example of a gene fusion event associated with cancer is EML4-ALK. The fusion EML4-ALK predominantly occurs in non-small cell lung cancer. When EML4 gene fuses with the kinase domain of ALK in lung cells, these cells experience abnormal signaling which results in increased cell proliferation and eventually cancer. Currently, EML4-ALK fusion is a biomarker for ALK inhibitors such as crizotinib, ceritinib or alectinib. Other cancer causing gene fusions include BCR-ABL1 in myelogenous leukemia, TMPRSS2-ERG in prostate cancer, PTPRK-RSPO3 in colorectal cancer and many more.

At the step “Check for Gene in GTF File,” 212, if a gene is not found in the GTF file, the software removes that fusion from the analysis and outputs a notification to the user.

At the step “Load Reference Genome,” 214, the standardized reference genome is loaded into memory. At the step “Check ‘-o’ Parameter,” 216, if the parameter “-o append” is applied, the gene fusions will be appended to a copy of the input reference genome. If the parameter “-o genes” is applied, the new reference genome will include all gene sequences from the GTF file and the fused gene sequences. Otherwise, only the fused genes and gene sequence of each gene in the gene fusion input file will be extracted from the standardized reference genome and outputted to the new customized reference genome, preferably under the “>Gene1_Gene2_fusion#” tag.

At the step “Check Chromosome in Reference Genome,” 218, the software checks if the chromosome associated with each gene in the gene fusion input file is present in the standardized reference genome. If any of the chromosomes do not match up, at “Terminate/Notification” 218, the software terminates and outputs a notification asking the user to find a matching GTF file and standardized reference genome. The software then proceeds to the step “Check Non-Altered Nucleotides,” 220.

At the step “Check Non-Altered Nucleotides,” 220, non-altered nucleotides in the gene fusion input file will be checked against the standardized reference genome to make sure they match. If they do not match, at “Terminate/Notification” 222, the software terminates and outputs a notification asking the user to find a matching GTF file and standardized reference genome.

At the step “Write WT and Fusion Sequences,” 224, the wild type sequence for each gene with a fusion and the fusion event, which is the merged version of two genes at a specific breakpoint, is written to a new customized reference file. The sequences in the new file will contain the gene name for wild type sequences (i.e. “>Gene1”) or a fused name for gene fusion sequences (i.e. “>Gene1_Gene2_fusion#”.

At the step “Generate Gene Fusion Index File,” 222, the software generates a fusion index file, which identifies where the wild type nucleotides and gene fusion events are located in the new customized reference genome. The customized reference genome, which identifies the gene fusion events, may then be used to diagnose cancers and other conditions that originate due to gene fusions and then treat those cancers with a pharmaceutically acceptable amount of an anti-cancer drug, as those that are known to those of ordinary skill in the art.

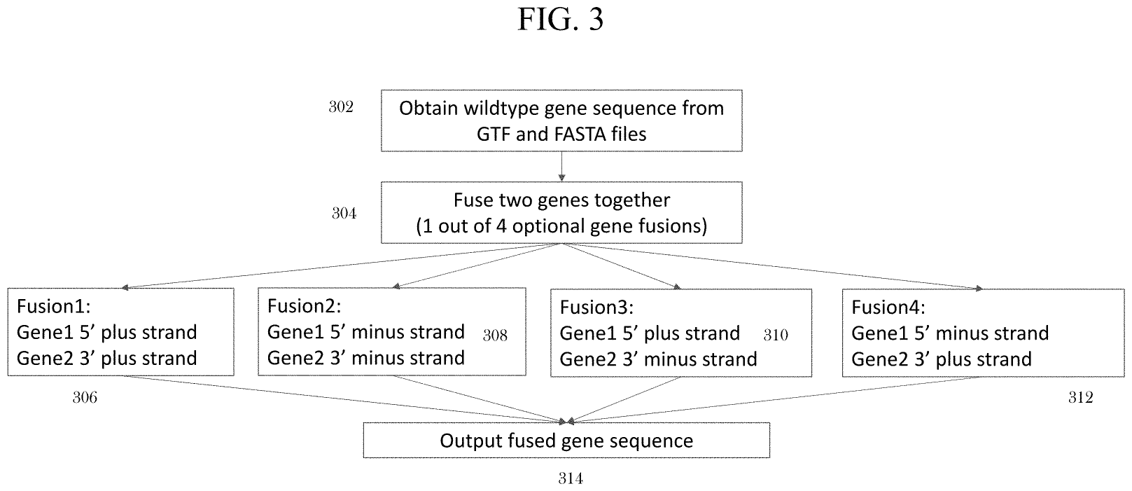

FIG. 3 is a flowchart detailing another exemplary process by which the software of the present invention operates in writing a gene fusion sequence. The process commences with the software obtaining wildtype gene sequences from GTF and/or FASTA databases 302. The software then fuses two genes together 304. The software has the option to modify the fused gene sequence with four types of fusions: 5′ plus+3′ plus=Gene1 [start:break]+Gene2[break:end] 306; 5′ minus+3′ minus=Gene2[start:break]+Gene1[break:end] 308; 5′ plus+3′ minus=Gene1[start:break]+Reverse(Gene2[start:break]) 310; and 5′ minus+3′ plus=Reverse(Gene2[break:end])+Gene1[break:end] 312. The software then outputs a fused gene sequence that includes the gene fusion events 314.

The software of the present invention has numerous applications. Customized reference genomes can be used to enhance detection of NGS reads containing a gene fusion, which can in turn improve matching cancer patients with the optimal therapeutic based on gene fusions present within their tumor. For example, approximately 95% of chronic myeloid leukemia patients and approximately 30% of acute lymphoblastic leukemia patients contain a BCR-ABL1gene fusion. The BCR-ABL1 fusion has shown to be a promising target in these patients and is used as a biomarker to guide treatment for tyrosine kinase inhibitors, which effectively inhibits the activity of the BCR-ABL1 protein. By using MAXX_Fusion on NGS data from these patients we can more confidently identify the presence of pre-defined BCR-ALB1 gene fusions, which will guide administration of tyrosine kinase inhibitors.

In other applications, customized gene fusion reference genomes can be used to improve the sensitivity of detecting circulating tumor cells and/or cell free tumor DNA/RNA that contain a gene fusion. Tumor DNA/RNA, either within a cell or cell free, is often at very low concentrations within the blood and is unique to individual patients. But with the use of MAXX_Fusion, we can create personalized reference genomes to enhance detection of tumor DNA/RNA containing a fusion event within a blood sample. The ability to detect tumor DNA/RNA within blood samples is an idea way to monitor previously treated cancer patients for cancer recurrence.

The foregoing description and drawings should be considered as illustrative only of the principles of the invention. All references cited herein are incorporated in their entireties. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All reference cited herein are incorporated by reference in their entirety.

1. A method of genetic analysis, comprising the steps of:
reading a plurality of input parameters, wherein the input parameters comprise a path to a gene fusion input file;
storing the gene fusion input file, wherein the gene fusion input file is comprised of a mutated genetic sequence comprised of one or more gene fusion events;
receiving data identifying chromosome location, start position, end position, and strand for each gene in the gene fusion input file;
loading a standardized reference genome;
comparing the gene fusion input file to the standardized reference genome; and
generating a gene fusion index file, wherein the gene fusion index file identifies gene fusion events in the customized reference genome, and wherein the gene fusion index file is used to diagnose a genetic condition.

2. The method of claim 1, wherein the gene fusion index file is used to quantify allelic expression.

3. The method of claim 1, wherein the genetic condition is a cancer.

4. The method of claim 1, further comprising requesting a new gene fusion input file if fusions in the gene fusion input file are duplicated.

5. The method of claim 1, wherein the comparison of the gene fusion input file to the standardized reference genome comprises matching non-altered nucleotides to an input reference genome.

6. The method of claim 1, wherein the customized reference genome file is comprised of wild type sequences and gene fusion sequences from the gene fusion input file or appended gene fusion sequences to the gene fusion input file or gene fusion sequences and all gene sequences of genes in the GTF file.

7. The method of claim 1, wherein the data identifying chromosome location, start position, end position, and strand for each gene in the gene fusion input file is in a GTF file format.

8. A genetic analysis system, wherein a computer:
reads a plurality of input parameters, wherein the input parameters comprise a path to a gene fusion input file;
stores the gene fusion input file, wherein the gene fusion input file is comprised of break points of two genes for one or more gene fusion events;
receives data identifying chromosome location, start position, end position, and strand for each gene in the gene fusion input file;
loads a standardized reference genome;
compares the gene fusion input file to the standardized reference genome; and
generates a gene fusion index file, wherein the gene fusion index file identifies the location of gene fusion events in the customized reference genome, and wherein the gene fusion index file is used to quantify the number or next generation sequencing reads aligned to the wild type allele and fused allele, wherein allelic expression of gene fusions is used to diagnose a genetic condition.

9. The system of claim 8, wherein the gene fusion index file is used to quantify the number of next generation sequencing reads aligned to the wild type allele or fused allele, wherein the quantification is performed using allelic expression of fusions.

10. The system of claim 9, wherein the genetic condition is diagnosed using the allelic expression of fusions.

11. The system of claim 8, wherein the genetic condition is a cancer.

12. The system of claim 8, wherein the computer further requests a new gene fusion input file if fusions in the gene fusion input file are duplicated.

13. The system of claim 8, wherein the comparison of the gene fusion input file to the standardized reference genome comprises matching non-altered nucleotides to an input reference genome.

14. The system of claim 8, wherein the customized reference genome is comprised of wild type sequences and gene fusion sequences from the gene fusion input file or only appended gene fusion sequences to the gene fusion input file or gene fusion sequences and all gene sequences of genes in the GTF file.

15. The system of claim 8, wherein the data identifying chromosome location, start position, end position, and strand for each gene in the gene fusion input file is in a GTF file format.
